(19) United States
(12) Patent Application Publication
(10) Pub. No.: US 2022/0047150 A1
(43) Pub. Date: Feb. 17, 2022

(54) SHEATH CAP AND ENDOSCOPE INTRODUCER SHEATH WITH SHEATH CAP

(71) Applicant: Wuhan Youcare Technology Co., Ltd., Wuhan (CN)

(72) Inventors: Shaogang WANG, Wuhan (CN); Xuecheng HU, Wuhan (CN); Yeyun MAO, Wuhan (CN); Xiao YU, Wuhan (CN); Wei GUAN, Wuhan (CN); Cao QIN, Wuhan (CN); Gang LONG, Wuhan (CN); Ying LI, Wuhan (CN); Chengpeng LIU, Wuhan (CN); Wenhua LI, Wuhan (CN)

(21) Appl. No.: 17/515,577

(22) Filed: Nov. 1, 2021

Related U.S. Application Data
(63) Continuation-in-part of application No. PCT/CN2019/097446, filed on Jul. 24, 2019.

(30) Foreign Application Priority Data
Apr. 30, 2019 (CN) 201910363388.4
Apr. 30, 2019 (CN) 201920624832.9

(51) Int. Cl. A61B 1/00; A61B 1/005; A61B 1/06; A61B 1/018; A61B 17/22

(52) U.S. Cl. CPC A61B 1/00135; A61B 1/0057; A61B 2217/007; A61B 1/018; A61B 17/22; A61B 1/063

(57) ABSTRACT

A sheath cap includes a housing collar and a cylinder which is coaxially fixed on the inner surface of one end of the housing collar. The cylinder is internally provided with instrument channel straight holes and a water spray channel straight hole which are parallel to the axial direction of the cylinder. The sheath cap is provided with an oblique hole, which is in communication with the water spray channel straight hole and is configured to spray water to the lateral front side of the sheath cap.

### CROSS-REFERENCE TO RELATED APPLICATIONS

This application is a continuation-in-part of International Patent Application No. PCT/CN2019/097446 with an international filing date of Jul. 24, 2019, designating the United States, now pending, and further claims foreign priority benefits to Chinese Patent Application No. 201910363388.4 filed Apr. 30, 2019, and to Chinese Patent Application No. 201920624832.9 filed Apr. 30, 2019. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the technical field of medical devices for human diseased tissues, and in particular to a sheath cap used for calculus in a human body cavity and an endoscope introducer sheath with the sheath cap.

At present, the surgical treatment of diseased tissues in the human body cavity has changed from traditional open surgery to minimally invasive surgery. The main method of minimally invasive surgery to treat calculi is to establish a channel, observe in real time under an endoscope, and perform lithotripsy and stone extraction operations with a holmium laser. The commonly used treatment device is an endoscope introducer sheath, and the endoscope introducer sheath introduces the holmium laser into human body through natural orifices to pulverize stones. For some stones that cannot be powdered, a stone basket may enter a kidney from an instrument passage to take granular stones out of the body. In the lithotripsy operation, it is necessary to inject water into the stone via a water spray joint of the endoscope introducer sheath, so that the broken calculi can be separated from the calculus point after being washed by the water, and thus the stone basket can be used to enter the instrument passage to take the broken calculi out or the broken calculi can be naturally expelled from body channels. However, due to the close connection between the broken calculi and the calculus point, the broken calculi are difficult to be expelled after being pulverized by the holmium laser, and are still attached to the calculus point, which affects the effect of the operation and also increases the pain of the patient.

### SUMMARY

An object of the disclosure is to overcome the above-mentioned prior art problems and provide a sheath cap which has a simple structure and can make a water flow effectively flush a calculus point to successfully expel broken calculi, and also an endoscope introducer sheath with the sheath cap.

To achieve the above object, a sheath cap of the disclosure comprises a housing collar and a cylinder which is coaxially fixed on an inner surface of one end of the housing collar. The cylinder is internally provided with instrument channel straight holes and a water spray channel straight hole which are parallel to an axial direction of the cylinder. The sheath cap is provided with an oblique hole, which is in communication with the water spray channel straight hole and is configured to spray water to the lateral front side of the sheath cap.

Based on this, the oblique hole can be designed into the following two structural forms:

Structural form 1: the oblique hole comprises inclined straight holes, each of which comprises one end communicating with the water spray channel straight hole and the other end extending to an outer surface of the housing collar on the side close to the front end face of the cylinder. The inclined straight holes are arranged along a circle of the sheath cap in the circumferential direction at intervals.

In an embodiment of the disclosure, the angle between the inclined straight hole and the water spray channel straight hole is 5°-85°.

Structural form 2: the oblique hole comprises water spray channel oblique slots, each of which is provided on the surface of the cylinder and forms an angle with the axial direction of the cylinder. A water spray connection channel is provided in the cylinder and is connected between the rear end of the water spray channel oblique slot and the water spray channel straight hole. The water spray channel oblique slots are provided on a surface of a circle of the cylinder in the circumferential direction at intervals.

In a class of this embodiment, the cylinder is coaxially fixed with a cylinder collar which makes a water flow be sprayed to the lateral front side of the sheath cap along the orientation of the water spray channel oblique slot, and the water outlet end of the water spray channel oblique slot is located in front of the cylinder collar.

In still another embodiment of the disclosure, the housing collar and the cylinder collar are coaxially and fixedly connected as a whole, and the outer diameter of the cylinder collar is the same as the outer diameter of the housing collar.

An endoscope introducer sheath with a sheath cap comprises an introducer sheath handle and an introducer sheath tube coaxially fixed on the front end of the introducer sheath handle, the introducer sheath tube is internally provided with an axial channel, the introducer sheath handle is provided with a plurality of instrument joints and a water spray joint, the instrument joints and the water spray joint are respectively provided along their respective axial directions with instrument joint channels and a water spray joint channel which are in communication with the axial channel of the introducer sheath tube. An inner surface of the other end of the housing collar is coaxially fixed on the front end of the introducer sheath tube, the instrument channel straight holes and the water spray channel straight hole are in communication with the axial channel of the introducer sheath tube.

In an embodiment of the disclosure, the introducer sheath tube comprises a rigid tube coaxially fixed on the front end of the introducer sheath handle and a flexible tube coaxially fixed on the front end of the rigid tube, and the other end of the housing collar is coaxially and fixedly connected onto the front end surface of the flexible tube, the housing collar is fixedly connected to a pull wire, and the pull wire passes through the axial channel of the introducer sheath tube to be connected to a control device for controlling the bending of the flexible tube, and the control device is provided on the introducer sheath handle.

In a class of this embodiment, the instrument joints comprise a holmium laser joint and an image illumination device joint, the holmium laser joint and the image illumination device joint are respectively provided along their respective axial directions with a holmium laser joint channel and an image illumination device joint channel which are in communication with the axial channel of the introducer sheath tube, the instrument channel straight holes comprise a holmium laser channel straight hole and an image illumination device channel straight hole. The holmium laser channel straight hole, the image illumination device channel straight hole and the water spray channel straight hole are in communication with the axial channel of the introducer sheath tube.

In still another embodiment of the disclosure, an axial partition is fixed in the introducer sheath tube, and the axial partition divides the axial channel of the introducer sheath tube into a first passage communicating with the holmium laser joint channel and a second passage communicating with the image illumination device joint channel and the water spray joint channel. The aperture of the second passage is larger than the aperture of the first passage. The first passage coaxially communicates with the holmium laser channel straight hole, and the second passage communicates with the image illumination device channel straight hole and the water spray channel straight hole.

The advantages associated with the disclosure are as follows: by designing the form of a combined hole structure combining the water spray channel straight hole and the oblique hole in the sheath cap, a water flow entering from the endoscope introducer sheath can be simultaneously sprayed to the front and the side of a calculus point in front of the sheath cap. While broken calculi at the calculus point are subjected to multi-angle washing to increase the probability of the broken calculi being removed from the calculus point, the front water flow and the side water flow of the sheath cap interact with each other to form a vortex, and the broken calculi is further separated from the calculus point under the influence of the vortex, thereby increasing the probability of separation between the broken calculi and the calculus point, and ensuring the washing effect of the water flow on the broken calculi. The endoscope introducer sheath is used in conjunction with the sheath cap. During the operation, the endoscope introducer sheath enters human body cavity under the introduction of the sheath tube, the image illumination device enters the front end of the endoscope introducer sheath via the image illumination device joint. The water spray joint is connected to a water pipe, and thus the water flow is sprayed out in the human body cavity through the oblique hole and the water spray channel straight hole, flushing the visual field to ensure that the image illumination device transmits a clear image. Operate the introducer sheath handle, move the endoscope introducer sheath back and forth, and adjust the bending angle of the introducer sheath tube in the cavity by the control device to find stones. After finding out the stone, holmium laser is provided via the holmium laser joint and is focused on the stone to crush it. The powdered stone is flushed out of the body through water circulation.

The sheath cap and the endoscope introducer sheath designed by the disclosure can effectively treat human calculus, and are easy to operate, greatly reduce the pain of patients, and have good therapeutic effects and high market application value.

### BRIEF DESCRIPTION OF THE DRAWINGS

FIG. 1 is a first perspective view of a sheath cap according to the disclosure;

FIG. 2 is a second perspective view of the sheath cap according to the disclosure;

FIG. 3 is a third perspective view of the sheath cap according to the disclosure;

FIG. 4 is a front view of the sheath cap according to the disclosure;

FIG. 5 is a top view of the sheath cap according to the disclosure;

FIG. 6 is a cross-sectional view of the sheath cap shown in FIG. 5 along the line A-A;

FIG. 7 is a perspective view of another sheath cap according to the disclosure;

FIG. 8 is an exploded view of the another sheath cap according to the disclosure;

FIG. 9 is a front view of the another sheath cap according to the disclosure;

FIG. 10 is a cross-sectional view of the sheath cap shown in FIG. 9 along the line B-B;

FIG. 11 is a perspective view of an endoscope introducer sheath with a sheath cap according to the disclosure;

FIG. 12 is a front view of the endoscope introducer sheath with a sheath cap according to the disclosure;

FIG. 13 is a cross-sectional view of the endoscope introducer sheath with the sheath cap shown in FIG. 12 along the line C-C; and

FIG. 14 is an enlarged view of the part marked by symbol D shown in FIG. 12.

Among them, 1—housing collar, 2—cylinder, 3—water spray channel straight hole, 4—oblique hole (4.1—inclined straight hole, 4.2—water spray channel oblique slot, 4.3—water spray connection channel), 5—cylinder collar, 6—introducer sheath handle, 7—introducer sheath tube (7.1—rigid tube, 7.2—flexible tube), 8 water spray joint, 9—water spray joint channel, 10—control device, 11—holmium laser joint, 12—image illumination device joint, 13—holmium laser joint channel, 14—image illumination device joint channel, 15—holmium laser channel straight hole, 16—image illumination device channel straight hole, 17—axial partition, 18—first passage, 19—second passage, 20—holmium laser connection tube, 21—water inlet connection tube, 22—image connection tube.

### DETAILED DESCRIPTIONS

The disclosure will be further described in detail below in conjunction with the drawings and specific embodiments.

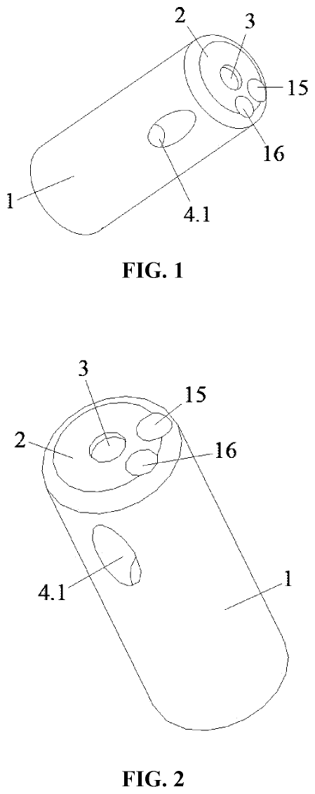

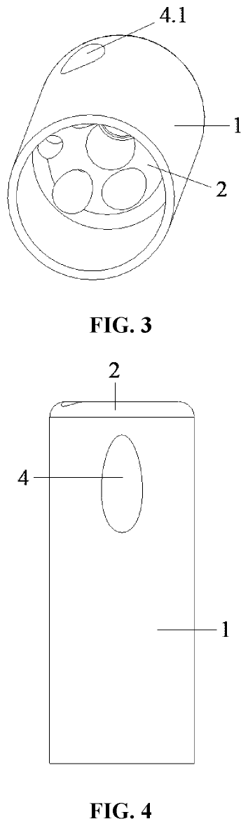

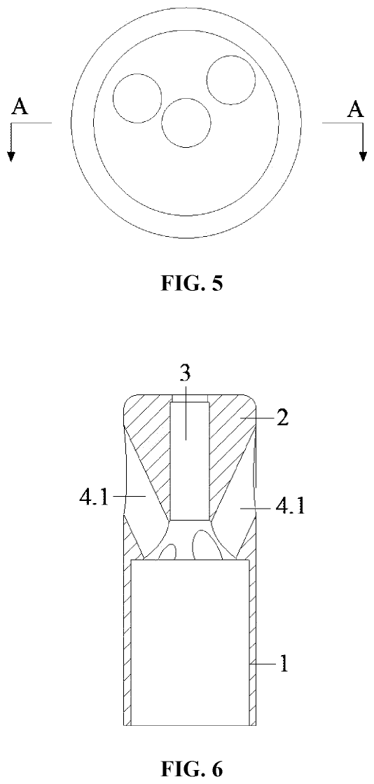

FIGS. 1-6 show an embodiment of a sheath cap, which comprises a housing collar 1 and a cylinder 2 which is coaxially fixed on an inner surface of one end of the housing collar 1. The cylinder 2 is internally provided with an image illumination device channel straight hole 16, a holmium laser channel straight hole 15 and a water spray channel straight hole 3 which are parallel to an axial direction of the cylinder 2. The sheath cap is provided with an oblique hole 4, which is in communication with the water spray channel straight hole 3 and is configured to spray water to the lateral front side of the sheath cap. The oblique hole 4 comprises inclined straight holes 4.1, each of which comprises one end communicating with the water spray channel straight hole 3 and the other end extending to a front end surface of the housing collar 1. The inclined straight holes 4.1 are symmetrically provided on the left and right sides of the sheath cap.

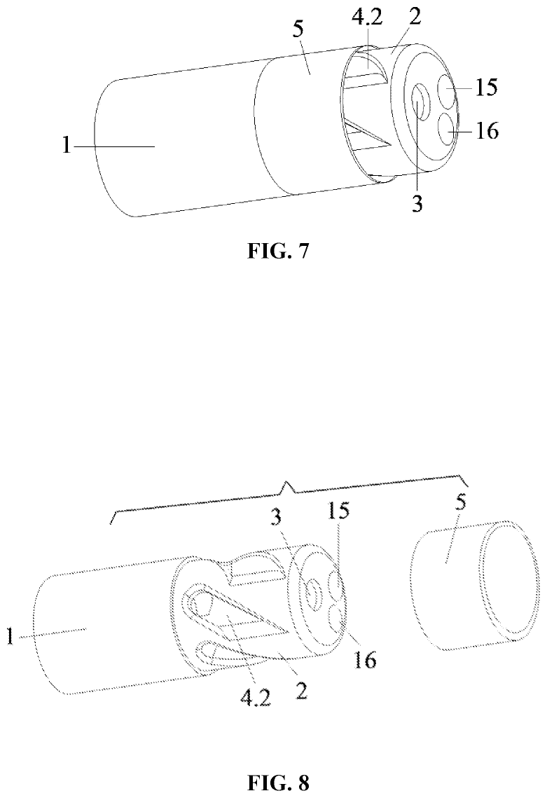

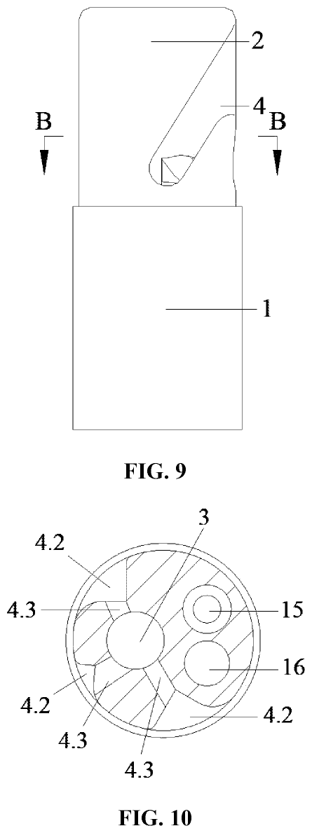

FIGS. 7-10 show another embodiment of a sheath cap, which comprises a housing collar 1 and a cylinder 2 which is coaxially fixed on an inner surface of one end of the housing collar 1. The cylinder 2 is internally provided with an image illumination device channel straight hole 16, a holmium laser channel straight hole 15 and a water spray channel straight hole 3 which are parallel to an axial direction of the cylinder 2. The sheath cap is provided with an oblique hole 4, which is in communication with the water spray channel straight hole 3 and is configured to spray water to the lateral front side of the sheath cap. The oblique hole 4 comprises water spray channel oblique slots 4.2, each of which is provided on the surface of the cylinder 2 and forms an angle with the axial direction of the cylinder 2. A water spray connection channel 4.3 is connected between the rear end of the water spray channel oblique slot 4.2 and the water spray channel straight hole 3 and is provided in the cylinder 2. The water spray channel oblique slots 4.2 are provided on the circumferential surface of the cylinder 2 at intervals. The cylinder 2 is coaxially fixed with a cylinder collar 5. The water outlet end of the water spray channel oblique slot 4.2 is located in front of the cylinder collar 5. The housing collar 1 and the cylinder collar 5 are coaxially and fixedly connected as a whole, and the outer diameter of the cylinder collar 5 is the same as the outer diameter of the housing collar 1.

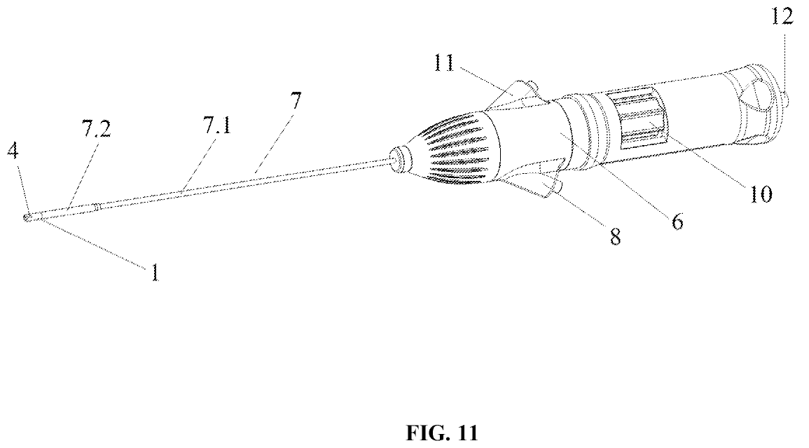

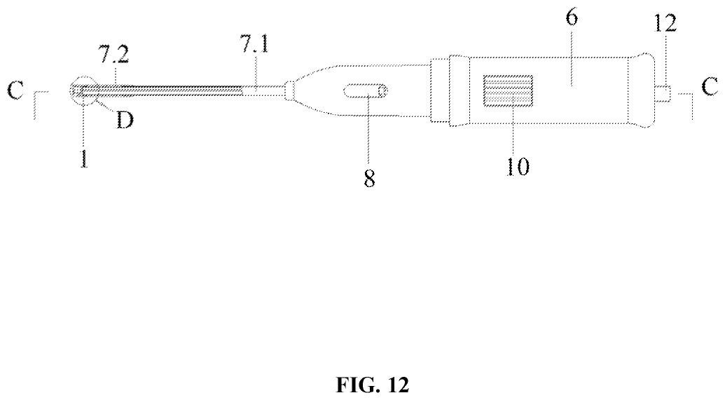

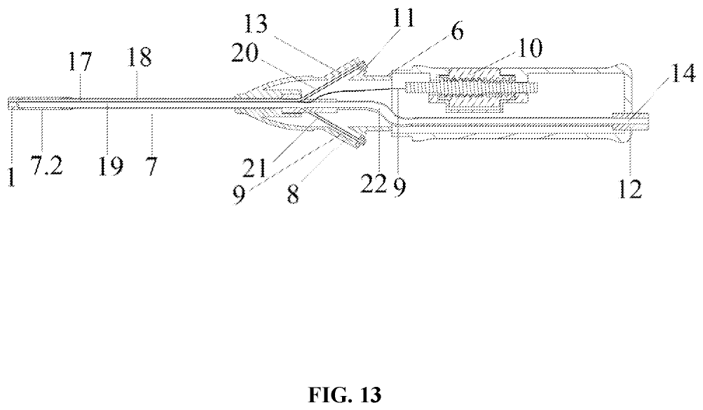

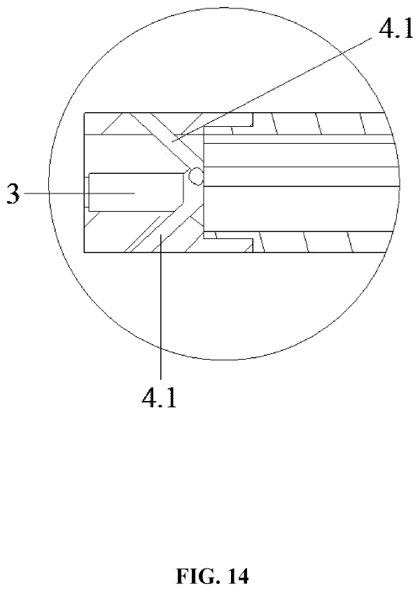

FIGS. 11-14 show an embodiment of an endoscope introducer sheath with a sheath cap, which comprises an introducer sheath handle 6 and an introducer sheath tube 7 coaxially fixed on the front end of the introducer sheath handle 6. The introducer sheath handle 6 is provided with a holmium laser joint 11, an image illumination device joint 12 and a water spray joint 8. The holmium laser joint 11, the image illumination device joint 12 and the water spray joint 8 are respectively provided along their respective axial directions with a holmium laser joint channel 13 and an image illumination device joint channel 14 and a water spray joint channel 9. The holmium laser joint channel 13 and the image illumination device joint channel 14 and the water spray joint channel 9 respectively communicate with an axial channel of the introducer sheath tube 7 through a holmium laser connection tube 20, an image connection tube 22 and a water inlet connection tube 21.

The introducer sheath tube 7 comprises a rigid tube 7.1 coaxially fixed on the front end of the introducer sheath handle 6 and a flexible tube 7.2 coaxially fixed on the front end of the rigid tube 7.1. The other end of the housing collar 1 is coaxially and fixedly connected onto the front end surface of the flexible tube 7.2. The housing collar 1 is fixedly connected to a pull wire 9, and the pull wire 9 passes through the axial channel of the introducer sheath tube 7 and is connected to a control device for controlling the bending of the flexible tube 10. The control device 10 is provided on the introducer sheath handle 6. A structure combining a thumb wheel and a thumb wheel lever could be used as the control device 10 of the disclosure. However, the disclosure is not limited as such, other structures such as a structure combining an activation lever and a spinning reel can also be used as the control device 10. By rotating the thumb wheel, the thumb wheel lever is driven to move forward and backward, thereby turning the pull wire 9 to move forward and backward in order to make the flexible tube 7.2 bend. An axial partition 17 is fixed in the introducer sheath tube 7, and the axial partition 17 divides the axial channel of the introducer sheath tube 7 into a first passage 18 communicating with the holmium laser joint channel 13 and a second passage 19 communicating with the image illumination device joint channel 14 and the water spray joint channel 9. The aperture of the second passage 19 is larger than the aperture of the first passage 18. The first passage 18 coaxially communicates with the holmium laser channel straight hole 15, and the second passage 19 communicates with the image illumination device channel straight hole 16 and the water spray channel straight hole 3.

In the disclosure, based on the holmium laser channel straight hole 15, the image illumination device channel straight hole 16 and the water spray channel straight hole 3, the oblique hole 4 is designed in the sheath cap, making the oblique hole 4 be in communication with the water spray channel straight hole 3. The water flow can be sprayed to the front and the lateral front side of the sheath cap when passing through the sheath cap and intending to be sprayed to the front of it. The water flow in two directions effectively strikes the broken calculi, and at the same time, the water flows in two directions interact with each other to form a vortex, which helps the floating and discharging of the broken calculi, thereby the disclosure has a simple structure and high practicality.

As shown in FIGS. 11-14, the endoscope introducer sheath is used in conjunction with the sheath cap. During the operation, the endoscope introducer sheath enters human body cavity under the introduction of the sheath tube, the image illumination device enters the front end of the endoscope introducer sheath via the image illumination device joint 12, and the water spray joint 8 is connected to a water pipe, and thus water flow is sprayed out in the human body cavity through the oblique hole 4 and the water spray channel straight hole 3, flushing the visual field to ensure that the image illumination device transmits a clear image. Operate the introducer sheath handle 6, move the introducer sheath handle 6 back and forth, and adjust the bending angle of the flexible tube 7.2 in the cavity by the control device 10 to find the stones. After finding out the stone, the holmium laser is provided via the holmium laser joint and is focused on the stone to crush it. The powdered stone is flushed out of the body through water circulation.

In the disclosure, the advantage of the endoscope introducer sheath used in conjunction with the sheath cap is that the introducer sheath tube 7 only has two passages, and there is only one wall between the passages, which can save space. The rigid tube 7.1 does not need to be bent, the wall thickness of rigid tube 7.1 can be thinner than that of the flexible tube 7.2, and the series of improvements enables the outer diameter of the rigid tube 7.1 to be much smaller (the flexible tube 7.2 and rigid tube 7.1 can have the same outer diameter to ensure the strength of the overall structure, or the flexible tube 7.2 can be thinner than the rigid tube 7.1 so that the flexible tube 7.2 can enter small cavities more easily, and the considerations can be selected according to the actual circumstances). During the operation, the endoscope introducer sheath carries the image illumination device, holmium laser and water flow into the kidney. Because the outer diameter of the rigid tube 7.1 is smaller than that of the flexible tube 7.2, the gap between the rigid tube 7.1 and the wall of the cavity is large, which is conducive for the water in the kidney to flow out quickly and thus the pressure in the kidney is lowered.

The sheath cap and the combination set of the sheath cap and the endoscope introducer sheath of the disclosure can effectively treat human calculus, and are easy to operate. The disclosure can greatly reduce the pain of patients, only one surgery can completely cure the stones in kidney calices and have good therapeutic effects and high market application value.

It will be obvious to those skilled in the art that changes and modifications may be made, and therefore, the aim in the appended claims is to cover all such changes and modifications.

What is claimed is:

1. A sheath cap, comprising a housing collar and a cylinder which is coaxially fixed on an inner surface of one end of the housing collar, the cylinder being internally provided with instrument channel straight holes and a water spray channel straight hole which are parallel to an axial direction of the cylinder, wherein the sheath cap is provided with an oblique hole which is in communication with the water spray channel straight hole and is configured to spray water to a lateral front side of the sheath cap.

2. The sheath cap of claim 1, wherein the oblique hole comprises inclined straight holes, each of which comprises one end communicating with the water spray channel straight hole and the other end extending to an outer surface of the housing collar on a side close to a front end face of the cylinder, and the inclined straight holes are arranged along a circle of the sheath cap in a circumferential direction at intervals.

3. The sheath cap of claim 2, wherein an angle between the inclined straight hole and the water spray channel straight hole is 5°-85°.

4. The sheath cap of claim 1, wherein the oblique hole comprises water spray channel oblique slots, each of which is provided on a surface of the cylinder and forms an angle with the axial direction of the cylinder; a water spray connection channel is provided in the cylinder and is connected between a rear end of the water spray channel oblique slots and the water spray channel straight hole, and the water spray channel oblique slots are provided on a surface of a circle of the cylinder in a circumferential direction at intervals.

5. The sheath cap of claim 4, wherein the cylinder is coaxially fixed with a cylinder collar so that a water flow is sprayed to the lateral front side of the sheath cap along an orientation of the water spray channel oblique slots, and a water outlet end of the water spray channel oblique slots is located in front of the cylinder collar.

6. The sheath cap of claim 5, wherein the housing collar and the cylinder collar are coaxially and fixedly connected as a whole, and an outer diameter of the cylinder collar is the same as an outer diameter of the housing collar.

7. An endoscope introducer sheath, comprising an introducer sheath handle and an introducer sheath tube coaxially fixed on a front end of the introducer sheath handle, the introducer sheath tube being internally provided with an axial channel, the introducer sheath handle being provided with a plurality of instrument joints and a water spray joint, the instrument joints and the water spray joint being respectively provided along their respective axial directions with instrument joint channels and a water spray joint channel which are in communication with an axial channel of the introducer sheath tube, wherein the endoscope introducer sheath further comprises the sheath cap of claim 1; an inner surface of the other end of the housing collar is coaxially fixed on a front end of the introducer sheath tube, and the instrument channel straight holes and the water spray channel straight hole are in communication with the axial channel of the introducer sheath tube.

8. The endoscope introducer sheath of claim 7, wherein the introducer sheath tube comprises a rigid tube coaxially fixed on the front end of the introducer sheath handle and a flexible tube coaxially fixed on the front end of the rigid tube, and the other end of the housing collar is coaxially and fixedly connected onto a front end surface of the flexible tube, the housing collar is fixedly connected to a pull wire, and the pull wire passes through the axial channel of the introducer sheath tube to be connected to a control device for controlling a bending of the flexible tube, and the control device is provided on the introducer sheath handle.

9. The endoscope introducer sheath of claim 8, wherein the instrument joints comprise a holmium laser joint and an image illumination device joint; the holmium laser joint and the image illumination device joint are respectively provided along their respective axial directions with a holmium laser joint channel and an image illumination device joint channel which are in communication with the axial channel of the introducer sheath tube; the instrument channel straight holes comprise a holmium laser channel straight hole and an image illumination device channel straight hole; and the holmium laser channel straight hole, the image illumination device channel straight hole and the water spray channel straight hole are in communication with the axial channel of the introducer sheath tube.

10. The endoscope introducer sheath of claim 9, wherein an axial partition is fixed in the introducer sheath tube, and the axial partition divides the axial channel of the introducer sheath tube into a first passage communicating with the holmium laser joint channel and a second passage communicating with the image illumination device joint channel and the water spray joint channel, an aperture of the second passage is larger than an aperture of the first passage, the first passage is coaxially communicating with the holmium laser channel straight hole, and the second passage communicates with the image illumination device channel straight hole and the water spray channel straight hole.
